(19) United States
(12) Patent Application Publication
(10) Pub. No.: US 2005/0276731 A1
(43) Pub. Date: Dec. 15, 2005

(54) Apparatus with changeable atmosphere

(76) Inventor: Chien-Chin Chen, Pingtung City (TW)

Correspondence Address: John S. Egbert;Harrison & Egbert, 7th Floor, 412 Main Street, Houston, TX, 77002, US

(21) Appl. No.: 10/867,125

(22) Filed: Jun. 14, 2004

(51) Int. Cl. A61L 9/12

(52) U.S. Cl. 422/123; 422/125

(57) ABSTRACT

An apparatus with changeable atmosphere comprises multiple atmosphere materials belonging to different categories, at least one atmosphere generator and a controller, the atmosphere generator providing the dissipation energies of the atmosphere materials. The dissipation times and intensities of the various atmosphere materials are manipulated by controlling the distances between the various atmosphere materials and the at least one atmosphere generator, or the operation intensity of the at least one atmosphere generator, thereby the function of changing atmospheres can be acquired.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention is related to an apparatus with changeable atmosphere, more specifically to an apparatus capable of changing, selecting or mixing atmospheres.

### BACKGROUND OF THE INVENTION

Nowadays, fragrance vaporizers for either natural or artificial perfumes are in wide use in families, offices or cars so as to remove odor. Because essences serving as the source of fragrance may have the curative effects of sedation, relaxation, reinvigoration or the like, numerous essences and the related apparatuses for air-freshening purposes have become popular in the current market.

Conventionally, volatilizing perfume materials such as essences can be implemented by heating. However, such perfume volatilization may incur olfaction inactivation after being conducted for a certain amount of time, and therefore the expected effect is diminished. Further, it is hard to change the smell to be of a different category during processing in accordance with conventional methods, so the conventional methods usually cannot satisfy the requirements in an environment in need of various atmospheres such as an office for changing atmosphere with time so as to harmonize working temper, a home for temperament and interest adjustment, or a fragrance curative place sequentially dissipating various atmospheres to harmonize human mental and physical conditions.

### BRIEF SUMMARY OF THE INVENTION

The objective of the present invention is related to an apparatus capable of changing, selecting and combining the sequences of dissipating atmospheres, and mixing atmospheres to meet the requirements of different demands and environments, so as to retard or thoroughly avoid the occurrence of olfaction inactivation.

The apparatus with changeable atmosphere of the present invention comprises multiple atmosphere materials belonging to different categories and at least one atmosphere generator, where the atmosphere generator is intended to provide the dissipation energy for the atmosphere materials. The times and intensities of various atmosphere material dissipations are manipulated by controlling the distance between the various atmosphere materials and the at least one atmosphere generator, or the operation intensity of the at least one atmosphere generator, thereby the function of changing atmospheres can be acquired.

The atmosphere generator capable of dissipating fragrances may be a flame of a candle or another flammable material capable of providing heat, and the atmosphere materials are individually positioned at different heights in respect of the candle or the flammable material. With the gradually lower flame of the candle, i.e., the distances between the flame and the atmosphere materials are shortened into volatilization activation regions sequentially, the dissipations of the atmosphere materials are activated in order.

Alternatively, the atmosphere generator may be a heater, and the various atmosphere materials are respectively put on a moveable mechanism as time goes on such as the hour hand and the minute hand of a clock. As a result, the various atmosphere materials can be sequentially dissipated at different times by rotating the hour hand and the minute hand.

Another apparatus with changeable atmosphere includes multiple various atmosphere materials, at least one atmosphere generator and a controller, where the at least one atmosphere generator provides energy to dissipate the atmosphere materials, and the controller controls the distance between the various atmosphere materials and the at least one atmosphere generator, and the operation intensity of the at least one atmosphere generator, so as to control the dissipation times and intensities of the atmosphere materials for changing atmospheres. In other words, the apparatus is a mechanism capable of controlling the positions of multiple various atmosphere materials to the atmosphere generator, and the atmospheres may be sequential, random or simultaneous dissipated, thereby the indoor atmosphere varies as time goes on.

Various atmosphere materials may be dissipated by the same atmosphere generator or exclusive atmosphere generators in respect of the various atmosphere materials. The former further comprises a moving apparatus to move the various atmosphere materials towards the atmosphere generator, and can be controlled by a controller to change atmospheres. The controller can control the ON/OFF switching and intensity of the atmosphere generator, so as to achieve abundant atmosphere changing effects.

### BRIEF DESCRIPTION OF THE SEVERAL VIEWS OF THE DRAWINGS

FIGS. 1(a) through 1(d) illustrate the apparatuses with changeable atmosphere of a candle type in accordance with the present invention;

FIGS. 1(e) through 1(g) illustrate other embodiments of the apparatus with changeable atmosphere in accordance with the present invention;

FIGS. 2(a) and 2(b) illustrate the apparatuses with changeable atmosphere of a clock type in accordance with the present invention;

FIGS. 2(c) and 2(d) illustrate the apparatuses with changeable atmosphere including rotating mechanisms in accordance with the present invention;

FIG. 3 illustrates an apparatus with changeable atmosphere with rotating atmosphere materials in accordance with the present invention; and

FIG. 4 illustrates a stationary apparatus with changeable atmosphere in accordance with the present invention.

### DETAILED DESCRIPTION OF THE INVENTION

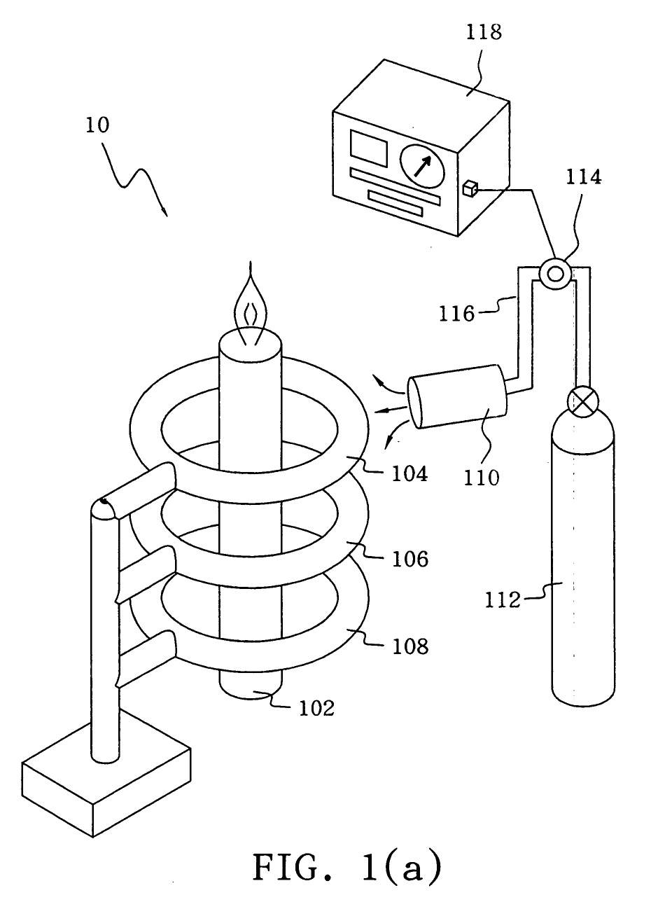

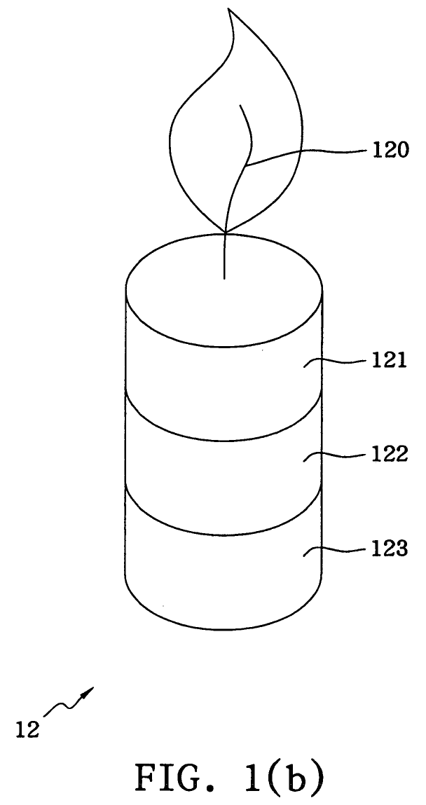

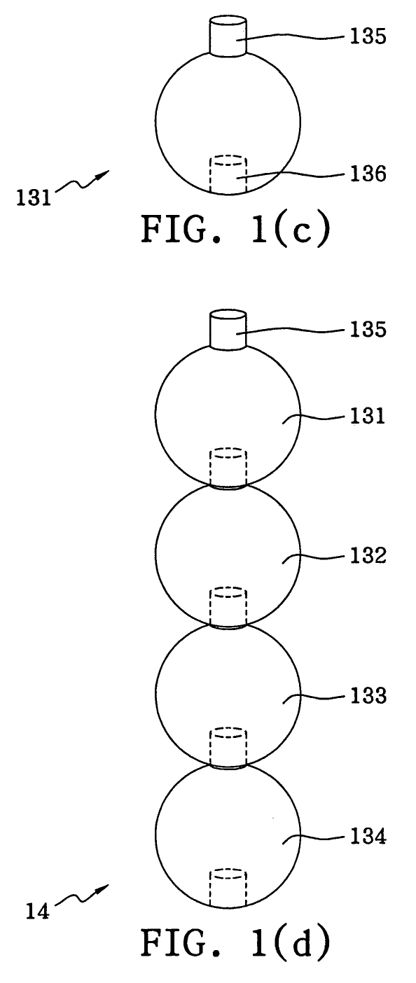

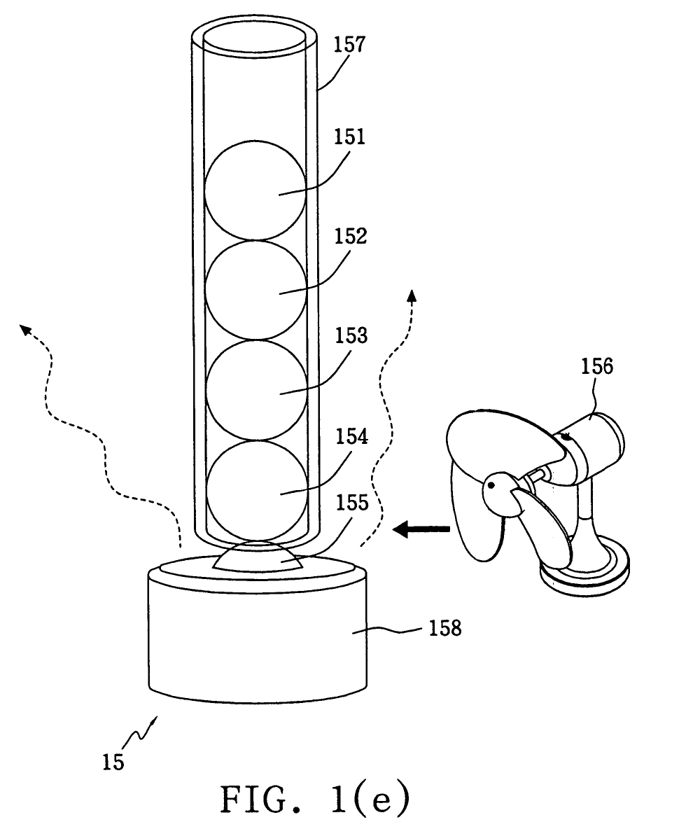

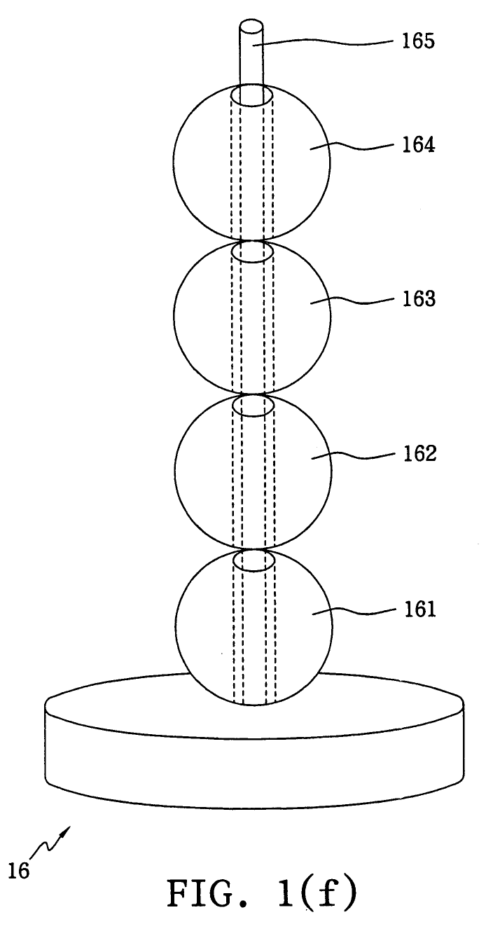

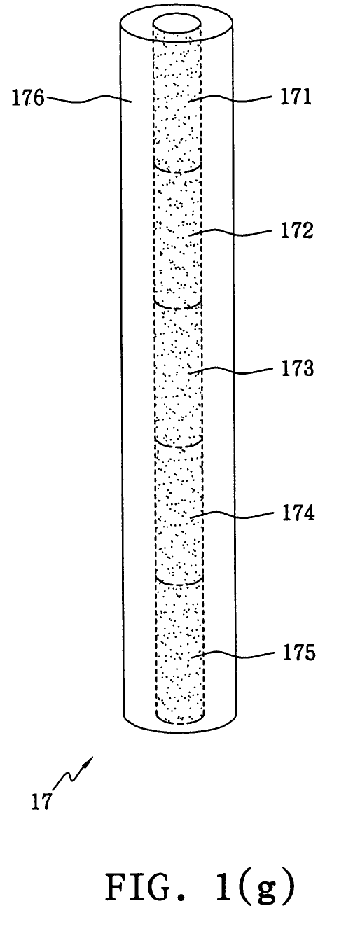

FIG. 1(a) illustrates an apparatus with changeable atmosphere in accordance with the present invention. An apparatus with changeable atmosphere 10 includes a candle 102, a first atmosphere material 104, a second atmosphere material 106, a third atmosphere material 108, an oxygen outlet 110, an oxygen bottle 112, a regulator 114, a gas hose 116 and a controller 118, where the first, second and third atmosphere materials 104, 106 and 108 are shaped as a ring and surround the candle 102 at different heights, and may be pure essences, materials absorbing perfumes or perfumes in containers. The oxygen outlet 110 faces the candle 102 and is connected to the oxygen bottle 112 through the gas hose 116, where the gas hose 116 is provided with a regulator 114 manipulated by the controller 118 to control oxygen flow. When the candle 102 is being flamed, the flame on the top of the candle 102 will gradually descend. Accordingly, when the flame is down to the height of the first atmosphere material 104, the first atmosphere material 104 will be dissipated by the heat of the flame. Likewise, when the flame is down to the second atmosphere material 106 and the third atmosphere material 108, atmospheres will be dissipated from the second and third atmosphere materials 106 and 108, respectively. In this embodiment, extra oxygen is introduced to enhance the burning efficiency so that dissipation timing can be well controlled.

FIG. 1(b) illustrates a changeable atmosphere candle 12 in which a wick 120 is surrounded by various atmosphere materials 121, 122, 123 serving as a wax candle body. As a result, the candle 12 can dissipate various atmospheres according to the different flame positions.

Referring to FIG. 1(c), an atmosphere ball 131 made of flammable materials is provided with a protrusion 135 and a hole 136 on the top and the bottom, respectively. The protrusion 135 conforms to the hole 136 for combination. As shown in FIG. 1(d), atmosphere balls 132, 133 and 134 are shaped as the ball 131 but composed of different atmosphere materials, and the four atmosphere balls 131, 132, 133 and 134 are together combined as an apparatus with changeable atmosphere 14 and burned in sequence to change atmosphere to be of different types.

Referring to FIG. 1(e), an apparatus 15 with changeable atmosphere comprises a heater 158, a fan 156, five atmosphere balls 151, 152, 153, 154 and 155 of different categories and a tube 157, where the atmosphere balls 151, 152, 153, 154 and 155 are vertically aligned in the tube 157 and disposed above the heater 155. When the atmosphere ball 155 is gradually dissipated out, the atmosphere ball 154 will be smaller and descended by gravity. Consequently, the atmosphere balls 155, 154, 153, 152 and 151 will be dissipated in order. Moreover, the fan 156 is intended to enhance the effectiveness of atmosphere dissipation.

Referring to FIG. 1(f), an apparatus 16 comprises four flammable atmosphere balls 161, 162, 163 and 164 are serially connected by a string 165, wherein the flammable atmosphere balls 161 to 165 are of different categories. As a result, if one of the flammable atmosphere balls is flamed, the atmosphere will be changed whenever the adjacent flammable balls are flamed also. The flammable atmosphere materials may be shaped as another type such as a plug, and can achieve the essentially equivalent effect.

As shown in FIG. 1(g), an apparatus 17 comprises five atmosphere materials 171, 172, 173, 174 and 175 connected in series, and a flammable layer 176 surrounds the five atmosphere materials 171 to 175. As a result, the atmosphere dissipation is based on the position where the flammable layer 176 burns.

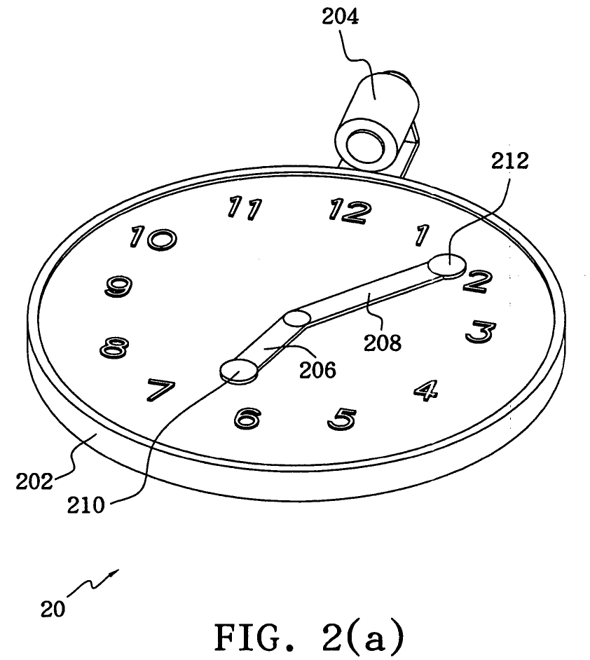

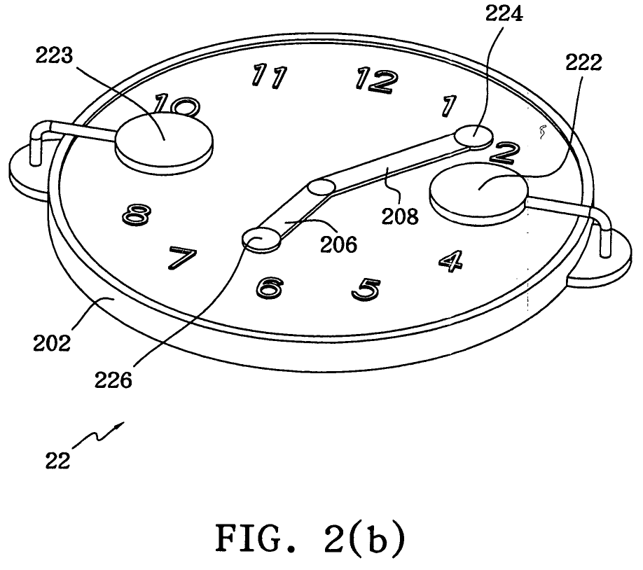

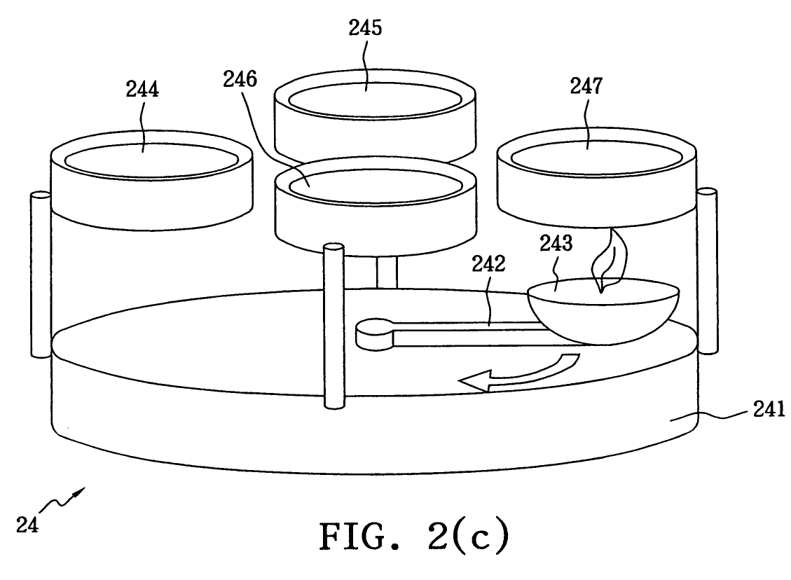

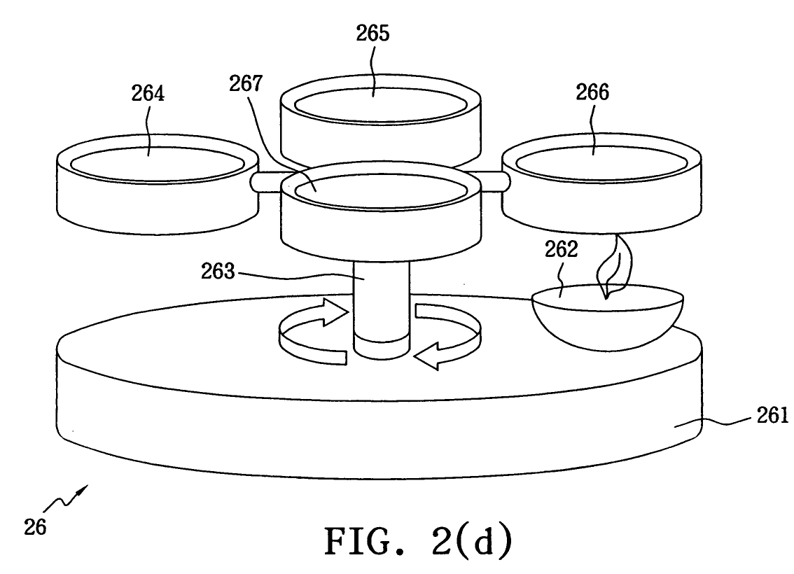

Referring to FIG. 2(a), an apparatus with changeable atmosphere 20 comprises a clock body 202, an hour hand 206, a minute hand 208, a atmosphere generator 204, a first atmosphere material 210 and a second atmosphere material 212, the first and second atmosphere materials being placed on the tips of the hour hand 206 and the minute hand 208. The atmosphere generator 204, e.g., a heater or the like capable of dissipating the first and second atmosphere materials 210, 212, is located at the “twelve o'clock” position of the clock body 202. The hour hand 206 and minute hand 208 rotate as time goes on, so the first and second atmosphere materials 210, 212 can be dissipated whenever the hour hand 206 and the minute hand 208 go through the “twelve o'clock” position, respectively. When the time is twelve o'clock, the hour hand 206 and the minute hand 208 are overlapped whereby a mixed atmosphere will be generated.

FIG. 2(b) illustrates another apparatus with changeable atmosphere 22 of clock type, in which heaters 226 and 224 are placed on the tip of the hour hand 206 and minute hand 208, respectively, whereas different atmosphere materials 222 and 223 are placed above the clock body 202 and close to the rotating paths of the hour hand 206 and the minute hand 208. Whenever the hour hand 206 or the minute hand 208 goes underneath the atmosphere material 222 or 223, the relevant atmosphere will be dissipated.

As shown in FIG. 2(c), an apparatus 24 comprises a rotating mechanism 241 and four atmosphere materials 244, 245, 246 and 247 of different categories. The rotating mechanism 241 is provided with a rotating rod 242 on the surface, and a heater, e.g., a candle 243, is disposed at the tip of the rotating rod 242. When the rotating rod 242 rotates, the candle 243 goes beneath the atmosphere materials 244, 245, 246 and 247 and therefore provides heat for dissipation.

As shown in FIG. 2(d), an apparatus 26 comprises a rotating mechanism 261 and four atmosphere materials 264, 265, 266 and 267 of different categories. The rotating mechanism 261 is provided with a heater such as a candle 262 on the surface and a rotating frame 263. The rotating flame 263 carries the four atmosphere materials 264, 265, 266 and 267, and thus the four atmosphere materials 264, 265, 266 and 267 go above the candle 262 in order for being dissipated when the rotating frame 263 rotates.

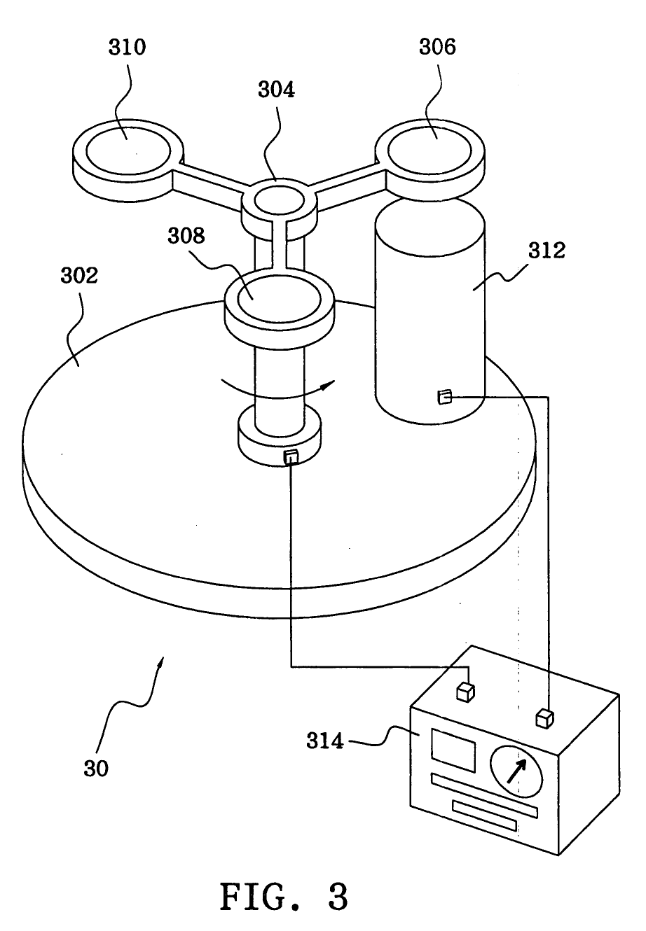

As shown in FIG. 3, an apparatus with changeable atmosphere 30 comprises a base 302, a rotation apparatus 304, a first atmosphere material 306, a second atmosphere material 308, a third atmosphere material 310, an atmosphere generator 312 and a controller 314. The first, second and third atmosphere materials 306, 308 and 310 are spaced by 120 degrees and carried by the rotation apparatus 304. The atmosphere generator 312 is placed on the base 302, and the controller 314 is in control of the atmosphere generator 312 and the rotation apparatus 304. When the first atmosphere material 306 is above the atmosphere generator 312, the first atmosphere material 306 will be dissipated. Likewise, the second and third atmosphere materials 308, 310 can also be rotated to the place above the atmosphere generator 312 by the rotation apparatus 304 for atmosphere dissipation. The rotation apparatus 304 can rotate continuously or by point-to-point manner. The continuous rotation is similar to the rotations of the hour and minute hands in the clock embodiment mentioned above, whereas the point-to-point rotation can continuously dissipate atmosphere for a while if desired. In addition to controlling the rotation apparatus 304, the controller 314 can also control the operation intensity of the atmosphere generator 312. For example, the higher temperature of the atmosphere generator 312 can increase the dissipation efficiency of the atmosphere material.

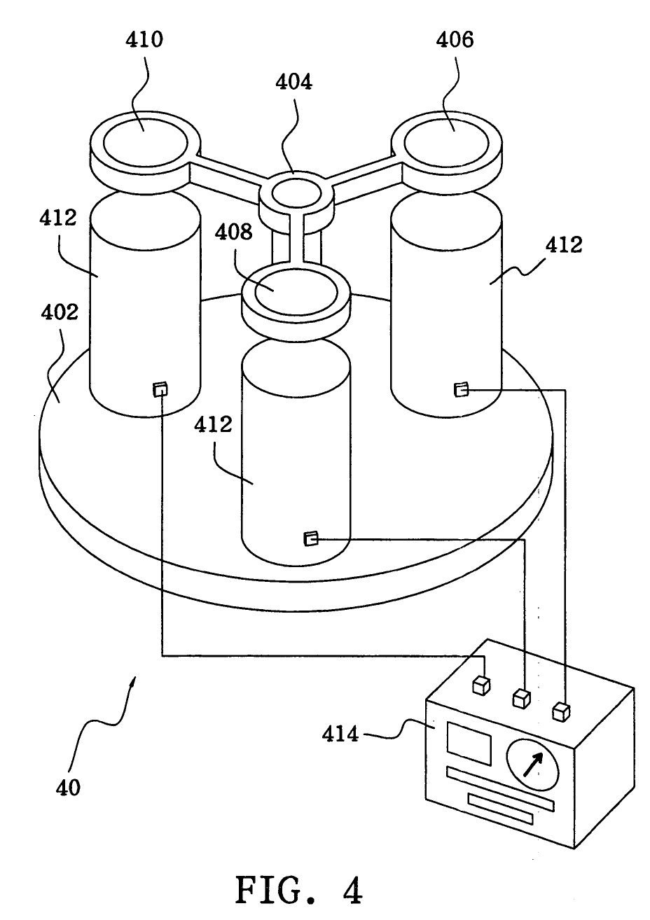

As shown in FIG. 4, an apparatus with changeable atmosphere 40 comprises a base 402, a support 404, a first atmosphere material 406, a second atmosphere material 408, a third atmosphere material 410, three atmosphere generators 412 and a controller 414. The first, second and third atmosphere materials 406, 408 and 410 are spaced by 120 degrees and supported by the support 404, and the three atmosphere generators 412 controlled by the controller 414 are respectively placed below the first, second and third atmosphere materials 406, 408 and 410. In comparison with the apparatus 30 shown in FIG. 3, the apparatus with changeable atmosphere 40 needs no rotation apparatus and the relevant control system so that the apparatus can be simplified. Further, two or three atmosphere generators 412 can be simultaneously activated to obtain mixed atmosphere that cannot be derived by the apparatus 30 in FIG. 3.

The above atmosphere materials may be essences of various categories, and the atmosphere generators 412 may be a heater of a high temperature type or a hot wind type to improve the dissipation of the atmosphere materials.

The above-described embodiments of the present invention are intended to be illustratively only. Numerous alternative embodiments may be devised by those skilled in the art without departing from the scope of the following claims.

1. An apparatus with changeable atmosphere, comprising:
a plurality of atmosphere materials belonging to different categories; and
at least one atmosphere generator for providing energy to dissipate the atmosphere materials;
wherein distances of the atmosphere materials and the one atmosphere generator are changeable so as to acquire capability of changing atmospheres.

2. The apparatus with changeable atmosphere in accordance with claim 1, wherein the atmosphere generator is comprised of a flame of a candle, and the atmosphere materials are placed at different heights in respect of the candle.

3. The apparatus with changeable atmosphere in accordance with claim 1, wherein the atmosphere materials are combined as a body of a candle.

4. The apparatus with changeable atmosphere in accordance with claim 3, wherein each of the atmosphere materials comprises a protrusion and a hole for assembly.

5. The apparatus with changeable atmosphere in accordance claim 1, wherein the atmosphere generator is a heater.

6. The apparatus with changeable atmosphere in accordance with claim 1, wherein the atmosphere materials belonging to different categories are placed on an hour hand and a minute hand of a clock.

7. The apparatus with changeable atmosphere in accordance with claim 1, wherein the one atmosphere generator is placed on an hour hand or a minute hand of a clock.

8. The apparatus with changeable atmosphere in accordance with claim 7, wherein the atmosphere materials are placed close to rotating paths of the hour hand and the minute hand.

9. The apparatus with changeable atmosphere in accordance with claim 1, wherein at least one of the atmosphere materials comprises essence.

10. The apparatus with changeable atmosphere in accordance with claim 1, wherein the atmosphere materials are descended when at least one of the atmosphere materials is dissipated.

11. The apparatus with changeable atmosphere in accordance with claim 1, wherein the atmosphere materials are flammable and connected in series.

12. The apparatus with changeable atmosphere in accordance with claim 1, wherein the atmosphere materials are connected in series and surrounded by a flammable layer.

13. The apparatus with changeable atmosphere in accordance with claim 1, wherein the atmosphere generator rotates close to the atmosphere materials in order for dissipation.

14. The apparatus with changeable atmosphere in accordance with claim 1, wherein the atmosphere materials rotate close to the atmosphere generator in order for being dissipated.

15. An apparatus with changeable atmosphere, comprising:
a plurality of atmosphere materials belonging to different categories;
at least one atmosphere generator providing energy to dissipate the atmosphere materials; and
a controller manipulating distances between the atmosphere materials and the one atmosphere generator, or controlling operation intensity of the one atmosphere generator, so as to control dissipation time and intensity of the atmosphere materials.

16. The apparatus with changeable atmosphere in accordance with claim 15, wherein the atmosphere generator is a heater.

17. The apparatus with changeable atmosphere in accordance with claim 15, wherein the atmosphere materials belonging to different categories are moveable and dissipated by a same atmosphere generator.

18. The apparatus with changeable atmosphere in accordance with claim 15, wherein the atmosphere materials belonging to different categories are dissipated by different atmosphere generators, respectively.

19. The apparatus with changeable atmosphere in accordance with claim 15, wherein at least one of the atmosphere materials comprises essence.

20. The apparatus with changeable atmosphere in accordance with claim 18, wherein at least two of the atmosphere materials are simultaneously dissipated so as to generate mixed atmosphere.
